(19) United States
(12) Patent Application Publication
(10) Pub. No.: US 2025/0262392 A1
(43) Pub. Date: Aug. 21, 2025

(54) ELECTRONIC DEVICE FOR COUNTING DISPENSABLE DOSES FROM A DEVICE FOR DISPENSING DOSES OF A MEDICINAL, COSMETIC, OR OTHER PRODUCT

(71) Applicant: FLEEP TECHNOLOGIES S.R.L., Milano (IT)

(72) Inventors: Giorgio DELL'ERBA, Milano (IT); Andrea PERINOT, Milano (IT); Paolo COLPANI, Milano (IT)

(21) Appl. No.: 18/038,889

(22) PCT Filed: Nov. 24, 2021
(86) PCT No.: PCT/EP2021/082859

§ 371 Date: Apr. 4, 2024

(30) Foreign Application Priority Data
Nov. 26, 2020 (IT) 102020000028571

(51) Int. Cl. A61M 15/00; B05B 11/00

(52) U.S. Cl. CPC A61M 15/008; A61M 15/009; B05B 11/0005; A61M 2205/13; A61M 2205/581; A61M 2205/583

(57) ABSTRACT

Electronic device (1) for counting dispensable doses from a device for dispensing doses of a medicinal, cosmetic or other product, comprising a flexible support substrate (2) to which an electronic control unit (3), a sensor unit (4) operatively connected to the electronic control unit (3), an information signaling unit (5) operatively connected to the electronic control unit (3), an electric power supply unit (6) operatively connected to the electronic control unit (3) are associated. The electronic control unit (3) is configured to detect an event connected to the dispensing of a dose, by the device for dispensing doses with which the device (1) is associated, based on the data provided by the sensor unit (4); performing a sequential count of the doses dispensed by the device for dispensing doses with which the device (1) is associated based on data representative of a user's interaction with the device for dispensing doses received from the sensor unit (4); record the sequential count performed; determine an information representative of the use of the device for dispensing doses based of the value achieved with the sequential count performed; provide the information signaling unit (5) with the determined information representative of the use of the device for dispensing doses.

### DESCRIPTION

### Field of the Invention

The present invention refers to an electronic device for counting the doses that can be delivered by a device for dispensing doses of a medicinal, cosmetic or other product.

### State of the Art

In the medical field, devices for dispensing doses of medicinal products are known which are used by patients with pathologies requiring, for example, treatments for inhalation of medicaments in the form of aerosols, gases, powders, sprays and so on.

In order to be able to monitor a therapy or simply to avoid running out of doses, it is very important to keep track of the inhaled doses, therefore the doses remaining inside the container.

To this end, it is known to install electronic devices on such dispensing devices that detect, for example, an action performed by a user or an environmental event (for example, a sound or a mechanical movement) or a measurement of physical quantities (for example, a flow of gas or light) when the dispensing device is operated to deliver a dose of a medicament and count the number of dispensed doses or the number of doses remaining inside the container.

However, such electronic devices are not free from defects.

First of all, the electronic components (microchips) installed externally to the dispensing device generally show characteristics of mechanical bending and resistance to very low stresses, making such an electronic device of poor mechanical flexibility.

Furthermore, this electronic device can have even higher manufacturing costs than the dispensing device itself.

Furthermore, the assembly costs of such an electronic device (and relative modification of the respective production line) can exceed the advantages of simple installation on the dispensing device of a traditional measuring device.

In addition, since many devices for dispensing doses of a medicinal product are of the “disposable” type, the problem of disposing of the electronic components present inside such an electronic device installed on a dispensing device is also very important, especially if these electronic components require special treatments for their correct disposal.

As is known, for example, the integration of electronic devices based on silicon or inorganic semiconductors, make any object on which they are affixed a special waste whose disposal is complex and expensive, which can contribute to making it unsustainable, from an economic point of view and/or environmental, the addition of electronic functions to common objects, including those of large consumption.

Therefore, the need is strongly felt to have available an electronic device for counting dispensable doses from a device for dispensing doses of a medicinal product that can be easily integrated into existing dispensing devices, has low costs and is easily disposable at the end of the life cycle of the medicinal product.

### SUMMARY

The object of the present invention is to devise and make available an electronic device for counting the dispensable doses from a device for dispensing doses of a medicinal, cosmetic or other product which allows to at least partially obviate the drawbacks mentioned above with reference to the known technique and which, in particular, can be easily integrated into existing devices for dispensing doses, has low costs and can be easily disposed of at the end of the life cycle of the product.

This object is achieved by a device according to claim 1.

Advantageous embodiments of said device are the subject of the dependent claims.

### BRIEF DESCRIPTION OF THE FIGURES

Further characteristics and advantages of the device according to the invention will result from the following description of preferred embodiment examples, given by way of non-limiting example, with reference to the attached figures, in which:

FIGS. 1a and 1b show, respectively, an example of a device for dispensing doses and a component thereof to which an electronic device for counting dispensable doses from a device for dispensing doses of a medicinal, cosmetic or other product, according to the present invention, can be applied;

FIGS. 2a and 2b illustrate, respectively, the example of the device for dispensing doses and its component of FIGS. 1a and 1b to which an electronic device for counting dispensable doses from a device for dispensing doses of a medicament, cosmetic or other product, according to an embodiment of the present invention, can be applied;

FIG. 3 illustrates by means of a block diagram an electronic device for counting dispensable doses from a device for dispensing doses of a medicinal, cosmetic or other product according to an embodiment of the present invention;

FIG. 4 illustrates by means of a block diagram an electronic device for counting dispensable doses from a device for dispensing doses of a medicinal, cosmetic or other product according to a further embodiment of the present invention;

FIG. 5 illustrates by means of a block diagram an electronic device for counting dispensable doses from a device for dispensing doses of a medicinal, cosmetic or other product according to a further embodiment of the present invention;

FIGS. 6, 7 and 8 each illustrate, by means of a circuit diagram, an electronic device for counting dispensable doses from a device for dispensing doses of a medicinal, cosmetic or other product according to a further embodiment of the present invention, and

FIG. 9 illustrates a component of a device for dispensing doses of a medicinal product to which an electronic device for counting dispensable doses from a device for dispensing doses of a medicinal, cosmetic or other product, according to a further embodiment of the present invention, is applied.

It should be noted that in the figures the same or similar elements will be indicated with the same numerical or alphanumeric references.

### DESCRIPTION OF SOME PREFERRED EMBODIMENTS

With reference now to the aforementioned figures, the numerical reference 1 indicates as a whole an electronic device for counting dispensable doses from a device for dispensing doses of a medicinal, cosmetic or other product, hereinafter also an electronic device 1, according to the present invention.

The device 100, as will be further detailed in the following according to different embodiments, is applicable to devices for dispensing doses.

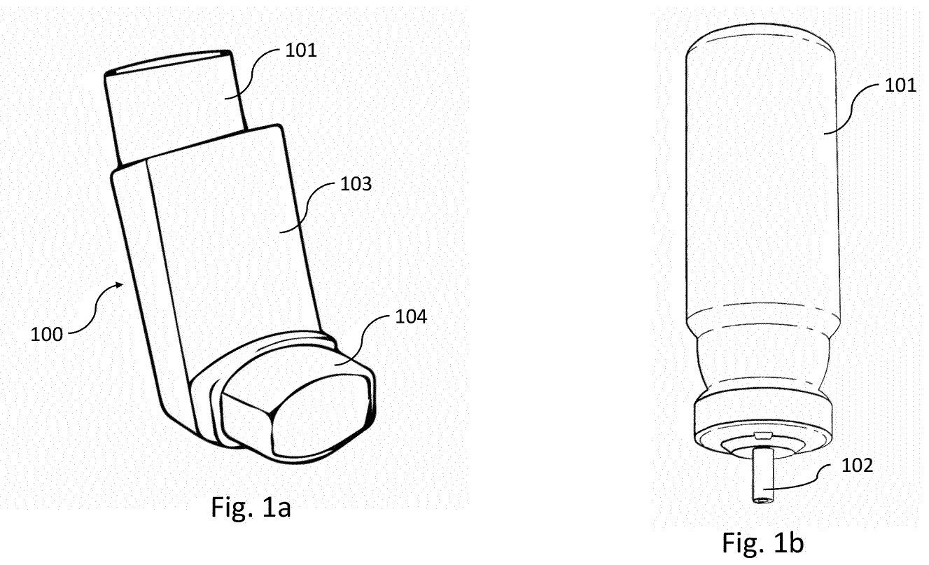

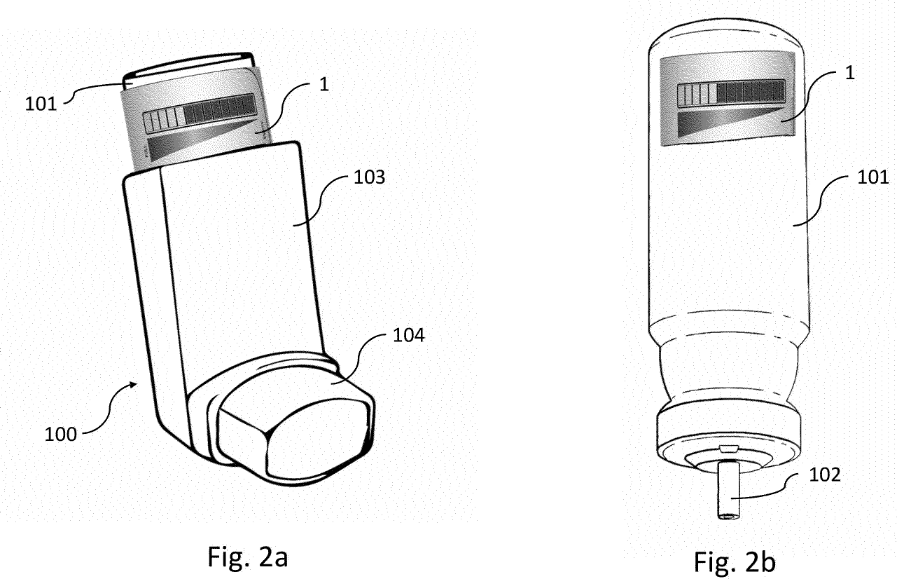

An example of a device for dispensing doses, indicated with the numerical reference 100, is illustrated in FIGS. 1a and 2a.

The device 100 for dispensing doses comprises a container (canister) 101 (illustrated separately in FIGS. 1b and 2b) inside which the product to be dispensed is present (not visible in the Figures).

The container 101, preferably cylindrical in shape, comprises at the base a valve 102 for dosing the product.

With reference again to FIGS. 1a and 2a, the device 100 for dispensing doses further comprises an actuator 103 equipped at one end with a mouthpiece 104.

The container 101 is housed inside the actuator 103 in such a way that a pressure from the top of the container 101 to push it inside the actuator 103 opens the valve 102 for dosing the product which emerges from the mouthpiece 104.

A device for dispensing doses to which the device 1 according to the present invention is applicable can be a dispenser of a medical product or drug, for example, a Metered Dose Inhaler (MDI, as the one shown in FIGS. 1a and 2a), a dry-powder inhaler or spray dispenser, a nebulizer or a vaporizer.

It should also be noted that the device 1 according to the present invention can be applied not only to a device for dispensing doses of a medical product but also to pressure-activated spray dispensers such as nebulizers, sprayers and vaporizers of a cosmetic product such as, for example, a cream or a deodorant, of high value and with a limited number of doses, in order to warn the user when the cosmetic product is close to the end.

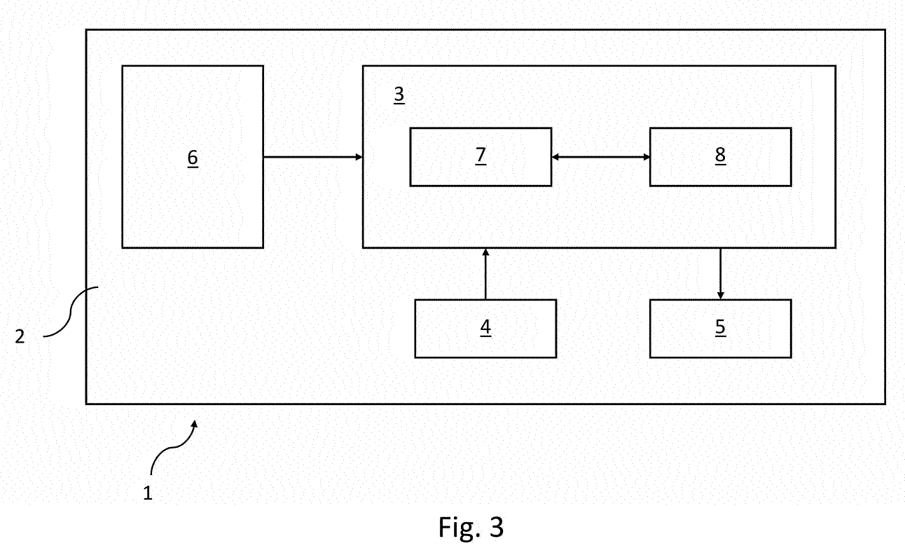

With reference to the diagram of FIG. 3, the device 1 comprises a flexible support substrate 2.

According to different embodiments, the flexible support substrate 2 can be composed of polymers and/or plastics and/or organic materials such as, for example, PET (Poly ethylene terephthalate), PEN (Poly ethylene naphthalate), Polyimide, paper.

In further embodiments, the flexible support substrate 2 can be composed of flexible materials such as flexible glass.

In further embodiments, the flexible support substrate 2 can be composed of bioplastics, for example Polylactic acid.

It should be noted that the flexibility characteristics of the flexible support substrate 2 are also dependent on its thickness.

The flexible support substrate 2 can have a thickness ranging from 1 micrometer to 200 micrometers, depending on the bending specifications required by the application and depending on the sustainable production cost for the same.

The flexible support substrate 2 has conformation properties, such as the possibility of being wrapped around objects with complex curvatures, such that it can be curved with a radius of curvature of about 3 cm, below 3 cm, below 1 cm, below 1 mm according to application specification.

With reference again to FIG. 3, the device 1 further comprises an electronic control unit 3, described in greater detail below, associated with the flexible support substrate 2.

According to an embodiment, also described below, in which the electronic control unit 3 is flexible, the electronic control unit 3 is associated with the flexible support substrate 2 by assembling said electronic control unit 3 to the flexible support substrate 2.

According to an embodiment, the electronic control unit 3 is manufactured, as will be reiterated below, with printing techniques comprising, but not limited to, printing techniques in so-called printed electronics technology, for example, ink jet printing, flexographic printing, screen printing, etching printing (gravure), in which the individual components are implemented with the superimposition of plastic materials and with manufacturing through the aforementioned printing techniques.

The device 1 also comprises a sensor unit 4, described in greater detail below, associated with the flexible support substrate 2.

The sensor unit 4 is operatively connected to the electronic control unit 3.

The device 1 further comprises an information signaling unit 5, described in greater detail below, associated with the flexible support substrate 2.

The information signaling unit 5 is operatively connected to the electronic control unit 3.

The device 1 further comprises an electric power supply unit 6 associated with the flexible support substrate 2.

The power supply unit 6 is operatively connected to the electronic control unit 3.

The device 1 further comprises connecting electrical interconnections associated with the flexible support substrate 2.

The electrical connection interconnections are made to electrically connect together the components of the device 1, listed above, in turn associated with the flexible support substrate 2.

The electronic control unit 3, the sensor unit 4, the information signaling unit 5, the power supply unit 6 and the relative electrical interconnections, like the flexible support substrate 2, are flexible, i.e. they have characteristics of thickness (for example up to a lower limit of 20 nanometers) and of manufacturing material that guarantee excellent mechanical flexibility properties.

Therefore, the device 1 advantageously represents a smart label of the “stand-alone” type.

In fact, the device 1 is independent of the need for elements, devices, processors, indicators or sources of energy external to it.

As will be described below, the device 1, as a label, can be applied to and removed from a device for dispensing doses, and thus reused several times with other devices for dispensing doses, up to the end of the life of the device 1.

According to an embodiment, the sensor unit 4, the information signaling unit 5, the power supply unit 6 and the relative electrical interconnections, like the electronic control unit 3, are associated with the substrate 2 flexible support by assembly.

According to an embodiment, like the electronic control unit 3, also the sensor unit 4, the information signaling unit 5, the power supply unit 6 and the related electrical interconnections are manufactured with printing techniques in so-called printed electronics technology, some examples of which have been previously provided, in which the individual components are implemented with the superimposition of plastic materials and with manufacturing through the aforementioned printing techniques.

With reference again to FIG. 3, the electronic control unit 3 comprises a control module 7 and a memory module 8 operatively connected to the control module 7.

The electronic control unit 3 is configured to detect an event connected to the dispensing of a dose, by the device for dispensing doses with which the device 1 is associated, based on the data provided by the sensor unit 4.

For the purposes of this description, “event connected to the dispensing of a dose” means the detection of a user action, for example pressing a button, or, alternatively, the detection of an environmental parameter connected to the dispensing of a dose, such as a flow of air or gas, a change in pressure, a change in humidity, heat, or a detection of specific chemical substance.

Furthermore, the electronic control unit 3 is configured to perform a sequential count (even by multiples of a set predetermined factor, in an increasing or decreasing manner) of the doses dispensed by the device for dispensing doses with which the device 1 is associated based on data representative of a user's interaction with the device for dispensing doses received from the sensor unit 4.

The sequential counting can be done incrementally or decrementally or alternately incremental and decremental.

According to various examples, the decrease can be equal to 700 units, less than 700 units, less than 200 units, less than 100 units, less than 10 units.

The electronic control unit 3 is configured to record the sequential count performed, for example, by storing a value corresponding to the sequential count reached in the memory module 8 or by changing an electronic status defined in the electronic control unit 3 or by causing a change of status of the electronic control unit 3 or other electronic components.

It should be noted that the recording of the sequential count can also be the storage in the memory module 8, for example in binary format, of the value reached with the sequential count performed by the electrical activation of the device 1, described below according to an embodiment, or in the activation of an electronic status univocally identified and connected to the value reached with the sequential count performed by the electrical activation of the device 1.

The electronic control unit 3 is configured to determine an information representative of the use of the device for dispensing doses based on the value achieved with the sequential count performed.

The electronic control unit 3 is also configured to provide the information signaling unit 5 with the determined information representative of the use of the device for dispensing doses.

For the purposes of this description, “information representative of the use of the device for dispensing doses” means information representative of a number of doses delivered by the device for dispensing doses, information representative of a number of remaining doses to be dispensed by the device for dispensing doses or information representative of a warning to indicate that the doses that can be dispensed by the device for dispensing doses are finishing or have finished (for example, reaching a minimum alarm threshold value of remaining doses).

In the event that the information representative of the use of the device for dispensing doses is the information representative of a number of doses dispensed by the device for dispensing doses, the electronic control unit 3 is configured to assign, to such information, the value achieved by the sequential count performed of the doses dispensed delivered by the device for dispensing doses.

In the event that the information representative of the use of the device for dispensing doses is the information representative of a number of remaining doses to be dispensed by the device for dispensing doses, the electronic control unit 3 is configured to determine such information as a difference between a maximum value of doses available inside the device for dispensing doses and the value achieved by the sequential count performed of the doses dispensed by the dose dispensing device.

In the event that the information representative of the use of the device for dispensing doses is a notification adapted to notify that the doses which can be delivered by the device for dispensing doses are finishing or have finished, the electronic control unit 3 is configured to determine such information by comparing a difference between a maximum value of doses available inside the device for dispensing doses and the value achieved by the sequential count of the doses dispensed by the device for dispensing doses with a minimum alarm threshold value of remaining doses.

It should be noted that the maximum value of doses available inside the device for dispensing doses can be stored in the memory module 8 of the electronic control unit 3 or in other memory modules with which the electronic control unit 3 can be equipped or by recording this value intrinsically to the design of the electronic control unit 3, for example by implementing an electronic circuitry configured to process a set maximum value of available doses inside the device for dispensing doses.

Alternatively, the maximum value of doses available inside the device for dispensing doses can be stored in the memory module 8, or in other memory modules (if present), which can be written or rewritten, after the device 1 has been manufactured.

In further embodiments, the electronic control unit 3 is configured to carry out further reading and/or writing operations in the memory module 8 and/or in further memory modules possibly present.

According to a further embodiment, the electronic control unit 3 is configured to amplify the operating voltage.

According to different embodiments, the thickness of the electronic control unit 3 can be 10 micrometers, less than 10 micrometers, less than 5 micrometers, less than 2 micrometers, less than 1 micrometer.

It should be noted that the mechanical flexibility properties of the electronic control unit 3, which consequently determine the maximum possible curvature of the device 1, are dependent on the overall thickness of the electronic control unit 3.

In this regard, the minimum radius of curvature for the electronic control unit 3 decreases as its overall thickness decreases.

It is therefore evident that the overall thickness of the electronic control unit 3 is a determining factor for obtaining the advantages of the present invention described above, in particular with reference to the purpose of being able to apply the device 1 in the form of a label on devices for dispensing doses commercially available, also characterized by surfaces with a radius of curvature less than one centimeter.

In accordance with the present invention, the electronic control unit 3 advantageously has an overall thickness of less than 10 micrometers.

In solutions belonging to the state of the art, in particular based on the application of microcontroller devices in silicon, the thickness characteristics and the mechanical characteristics of the silicon do not allow to obtain the application advantages obtainable with the electronic control unit 3 in accordance with present invention.

In this regard, it should also be noted that the thickness of the electronic control unit 3 is the fundamental limitation with respect to the thickness of the flexible support substrate 2 in terms of flexibility of the overall device 1.

In fact, since the flexible support substrate 2 has a purely structural support function, it can be chosen of an appropriate thickness with respect to the specifications dictated, for example, by considerations relating to manufacturing, for example also with a thickness of 1 micrometer, thus contributing in a secondary way to the determination of the maximum flexibility of the overall device 1.

From a circuit point of view, the electronic control unit 3 is a flexible integrated circuit comprising electronic elements including, but not limited to, transistors, capacitors, diodes, resistors, memory or data-storage elements.

In one embodiment, such integrated circuit (electronic control unit 3) can contain one or more transistors made in a thin film configuration (Thin Film Transistor—TFT) also made with organic materials (Organic Thin Film Transistors—OTFT).

OTFTs, in turn, can be made using only carbon-based materials including, for example, polymeric semiconductors, semiconductors based on small molecules, semiconductor carbon nanotubes or materials that integrate them.

These materials can be deposited from the liquid phase (from solution) also through the use of printing techniques comprising, but not limited to, printing techniques in printed electronics technology, for example, ink jet printing, flexographic printing, screen printing, etching printing (gravure).

TFTs can also be made using semiconductors based on metal oxides (e.g. Indium Gallium Zinc Oxide).

These materials can be deposited from the liquid phase (from solution) also through the use of printing techniques comprising, but not limited to, printing techniques in printed electronics technology (in English, “printed electronics”), for example, printing inkjet, flexographic printing, screen printing, etching printing (gravure).

In further embodiments, such integrated circuit (electronic control unit 3) can also comprise one or more organic electrochemical transistors (OECT), one or more transistors with capacitive control based on electrolyte (Electrolyte Gated Organic Field Effect Transistor—EGOFET), one or more vertical charge transport transistors.

As mentioned above, these components can also be made through the use of printing techniques comprising, but not limited to, printing techniques in so-called printed electronics technology, for example, printing inkjet, flexographic printing, screen printing, etching printing (gravure).

In some embodiments, such integrated circuit (electronic control unit 3) can also comprise one or more diodes.

These components can be made using only carbon-based materials including, for example, polymeric semiconductors, semiconductors based on small molecules, semiconductor carbon nanotubes or materials that integrate the same.

These materials can be deposited from the liquid phase (from solution) also through the use of printing techniques comprising, but not limited to, printing techniques in printed electronics technology, for example, inkjet printing, flexographic printing, screen printing, etching printing (gravure).

Furthermore, in some embodiments, such integrated circuit (electronic control unit 3) can comprise passive electronic components such as resistors, capacitors, inductors and memristors.

These components can be made with conductive materials even carbon-based only as polymeric conductors (e.g. PEDOT: PSS) or even nanoparticles-based (e.g. silver, copper nanoparticles).

Such passive electronic components may comprise carbon-based insulating materials (e.g. poly methyl methacrylate).

These materials can be deposited from the liquid phase (from solution) also through the use of printing techniques comprising, but not limited to, printing techniques in printed electronics technology, for example, inkjet printing, flexographic printing, screen printing, etching printing (gravure).

Furthermore, in some embodiments, such integrated circuit (electronic control unit 3) can comprise a combination of transistors, diodes and passive components, as described above, such as to create a circuit for analyzing the signal coming from the sensor unit 4.

In further embodiments, such integrated circuit (electronic control unit 3), in some embodiments, can contain a combination of transistors, diodes and passive components, as described above, such as to form a circuit that provides the drive signals for actuators.

In some embodiments, the integrated circuit (electronic control unit 3) can comprise a combination of transistors, diodes and passive components, as described above, such as to form a circuit for reading and/or writing memory elements.

In some embodiments, the integrated circuit (electronic control unit 3) may contain architectures for amplifying the operating voltage.

As regards the step of reading the data coming from the sensor unit 4, by the electronic control unit 3, it should be noted that the integrated circuit is configured to read the electrical signals arriving from the sensor unit 4 in order to recognize an interaction by the user with the device for dispensing doses with which the device 1 is associated.

In this regard, according to different embodiments, the reading component of the integrated circuit can also comprise at least one amplification circuit, at least one analog-digital converter, at least one recognition circuit for an electric voltage or electric current threshold, at least one operational amplifier, at least one differential amplifier.

These components can also be made through the use of printing techniques including, but not limited to, printing techniques in printed electronics technology, for example, inkjet printing, flexographic printing, screen printing, etching printing (gravure).

With reference now to the sensor unit 4, it is configured to detect data representative of an interaction by a user with the device for dispensing doses with which the device 1 is associated, connected to the dispensing of a dose.

As previously stated, this interaction is interpreted by the electronic control unit 3 to establish whether or not an event connected to the dispensing of a dose by the device for dispensing doses has occurred.

The sensor unit 4 comprises one or more sensors such as, for example, push buttons, touch sensors, photosensors, heat sensors.

Such one or more sensors can be arranged in matrix or segment configurations, or isolated.

As previously mentioned, the sensor unit 4 is operatively connected, through a respective electrical interconnection, to the electronic control unit 3.

In this embodiment, the sensor unit 4 is configured to receive electrical power directly from the electronic control unit 3.

In an embodiment, alternative to the previous one, the sensor unit 4 is operatively connected, through a respective electrical interconnection, to the electrical power supply unit 6.

It should be noted that the detection by the sensor unit 4 and the subsequent communication with the electronic control unit 3 can take place on the basis of a continuous, discrete, single-threshold or multiple-threshold electrical signal.

In one embodiment, the sensor unit 4 can comprise a single sensor, for example a pressure sensor with double threshold (a lower threshold and an upper threshold), suitably positioned on the device for dispensing doses.

In this embodiment, if the user exerts a pressure with sufficient force to exceed the upper threshold set in the double threshold pressure sensor, the electronic control unit 3 is configured to interpret the event as the dispensing of a dose to be counted.

If, on the other hand, the user exerts a pressure with sufficient force to exceed the lower threshold set in the double threshold pressure sensor but not to reach the upper threshold set in the double threshold pressure sensor, the electronic unit control 3 can be configured to interpret the event as a request by the user to know the value reached by the sequential count of the dispensed doses.

Therefore, in this case, the electronic control unit 3 can be configured not to interpret this event as a dispensed dose to be counted.

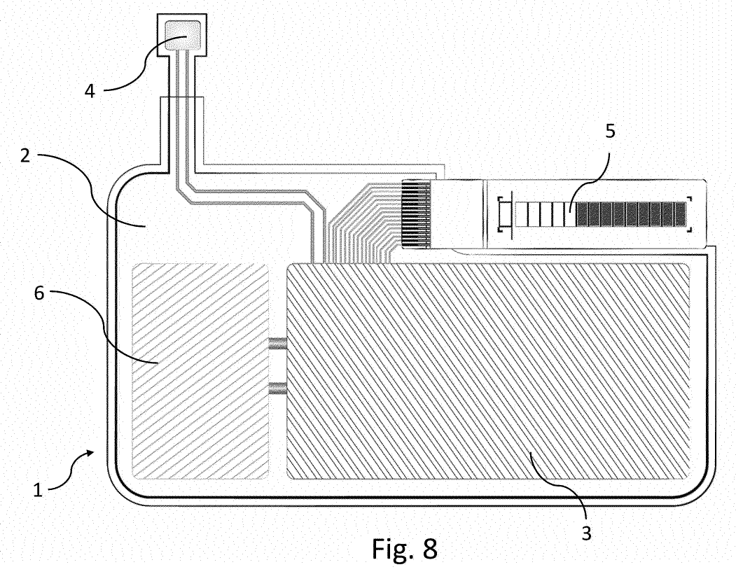

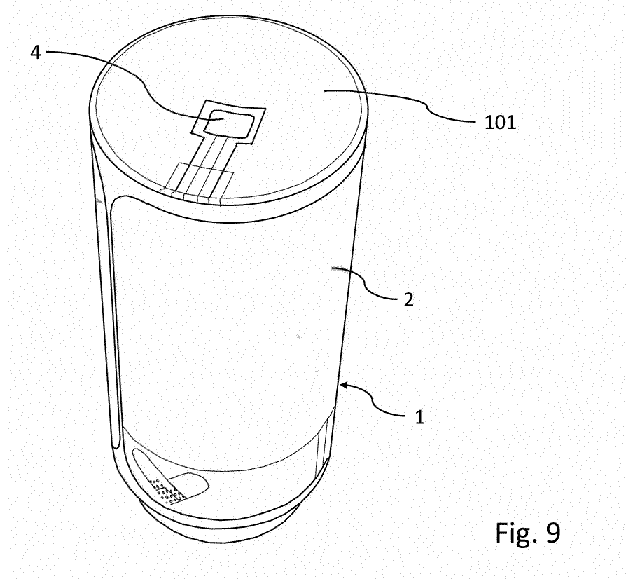

An example of such a pressure sensor, indicated with the numerical reference 4, is represented in FIGS. 8 and 9 in which embodiments of a device 1 according to the present invention are illustrated.

The example of pressure sensor 4 of FIG. 8 is represented in FIG. 9 in which the device 1, in the embodiment shown in FIG. 8, is applied to a container 101 of a device for dispensing doses (the latter not shown in the figures).

In FIG. 9, the pressure sensor 4 is applied to the center of the upper base of the container 101, that is, in the point where a user exerts pressure to obtain the delivery of a dose.

It should be noted that the pressure sensor 4 is associated with the flexible support substrate 2 through a respective electrical interconnection of such length that the pressure sensor 4 represents an appendage of the flexible support substrate 2.

In this way, as shown in FIG. 9, the device 1 is applicable to a container 101 of a device for dispensing doses in such a way that the flexible support substrate 2 is applied to the side wall of the container 101 while the pressure sensor is applied to the center of the upper base of the container 101.

According to a further embodiment, in which the sensor unit 4 comprises at least two sensors (also of different types), the sensor unit 4 is configured to detect actions or gestures of the user.

In an exemplary case, a first sensor (e.g., a photosensor) may be positioned on one side of a container (canister) of a device for dispensing doses (e.g., an MDI inhaler) while a second sensor (e.g., always a photosensor) can be positioned in a different position with respect to the first sensor, for example on the bottom of the same container (canister).

In this case, the electronic control unit 3 is configured to interpret the activation of the first (lateral) sensor as an action representative of grasping the inhaler and the activation of the second sensor (on the bottom) as an action representative of a dispensing of a dose.

In another exemplary case, a first sensor and a second sensor (for example, photosensors) can be positioned on the container (canister) in line at a distance, for example, of 1 cm from each other.

This arrangement advantageously allows to detect gestures such as a “sliding” of a finger, through the detection of a sequence comprising the activation of the first sensor and the subsequent activation of the second sensor.

Returning in general to the sensor unit 4, it should be noted that it can be achieved through the use of printing techniques comprising, but not limited to, printing techniques in printed electronics technology, for example, inkjet printing, flexographic printing, screen printing, etching printing (gravure).

With reference now to the information signaling unit 5, it is configured to provide the user with the information representative of the use of the device for dispensing doses.

In one embodiment, the information signaling unit 5 comprises a display unit.

By way of example, the display unit can comprise light emitting devices (LEDs, OLEDs,), electrochromic, thermochromic or electrophoretic devices.

In a further embodiment, alternatively or in combination with the previous one, the information signaling unit 5 can comprise at least one device for triggering a mechanical movement, for example vibrating or mobile membranes, or at least one sound emitter device.

The actuators can be arranged in matrix or segment configurations or isolated.

The supply of the information representative of the use of the device for dispensing doses can take place, by the signaling unit 5, continuously (for example with the luminous indicator constantly lit) or repeatedly at constant intervals (for example, always with flashing indicator light) or only when the dose is dispensed or at the request of the user, as will be described below in accordance with an embodiment.

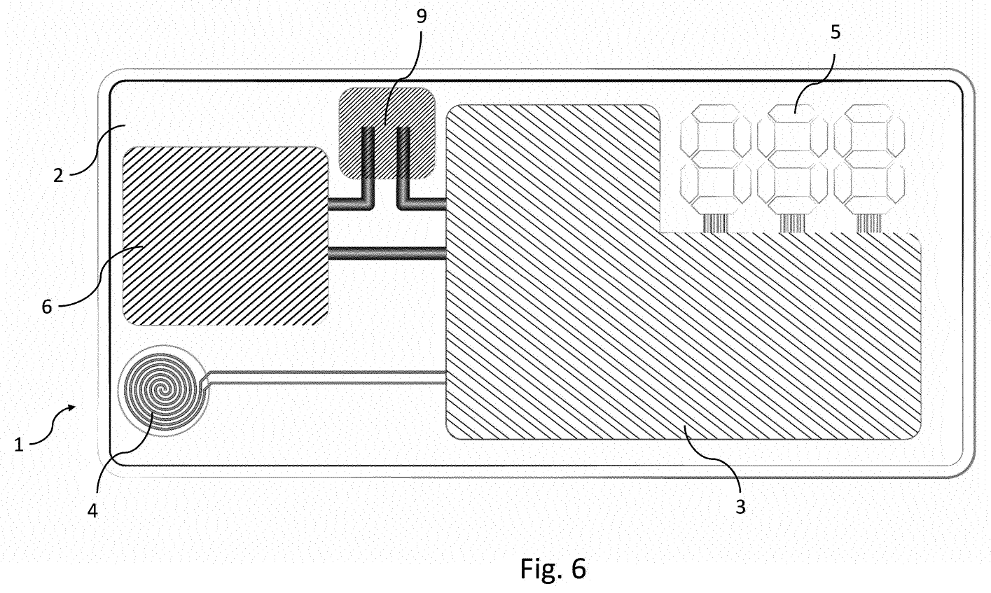

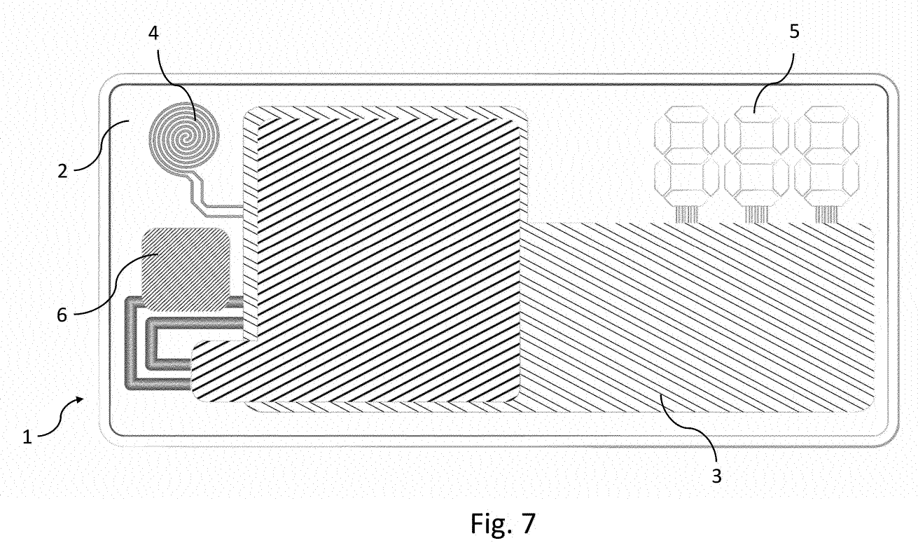

The representation of the information representative of the use of the device for dispensing doses can be carried out through a series of elements arranged according to a set configuration, for example with a segment display (see for example FIGS. 6 and 7), possibly of a suitable shape to represent significant symbols, or through a set arrangement of light-emitting elements.

The signaling status of these elements can be updated at each update of the sequential count, or at regular intervals set within the sequential count.

For example, it is possible to represent a progress bar indicating the number of doses remaining at intervals of five doses (see for example FIGS. 2a, 2b and 8) or it is possible to update the signaling status of the light emitting elements at regular time intervals (e.g. to generate a flashing effect).

It should be noted that also the information signaling unit 5 can be realized through the use of printing techniques comprising, but not limited to, printing techniques in printed electronics technology, for example, inkjet printing, flexographic printing, screen printing, etching printing (gravure).

With reference now to the electric power supply unit 6, in one embodiment, such unit can comprise energy accumulators, such as for example one or more primary or secondary batteries or supercapacitors.

In a further embodiment, the power supply unit 6 can comprise further energy-harvesters such as, for example, photovoltaic cells, thermoelectric generators, triboelectric generators.

In a further embodiment, the power supply unit 6 can also be a combination of two or more of the components provided in the previous embodiments.

In a further embodiment, in combination with any of those described above, the power supply unit 5 comprises a control circuitry for the dynamics of electric current supply: for example, electric voltage and/or discharge regulators downstream of the power supply unit 6.

In a further embodiment, in combination with any of those described above, the power supply unit 6 can comprise a recharge control circuitry of the power supply unit 6, configured to manage, for example, the recharging of an electrical power supply unit consisting of a rechargeable battery and one or more energy harvesters.

According to a further embodiment, in combination with any of those described above, the power supply unit 6 can further comprise an antenna for collecting energy from electromagnetic radiation, a rectifier (for example, composed with one or more diodes printed or with transistors, as described above, in architecture, for example, with transdiode) and a capacitor.

It should be also noted that the power supply unit 6 can be realized through the use of printing techniques comprising, but not limited to, printing techniques in printed electronics technology, for example, inkjet printing, flexographic printing, screen printing, etching printing (gravure).

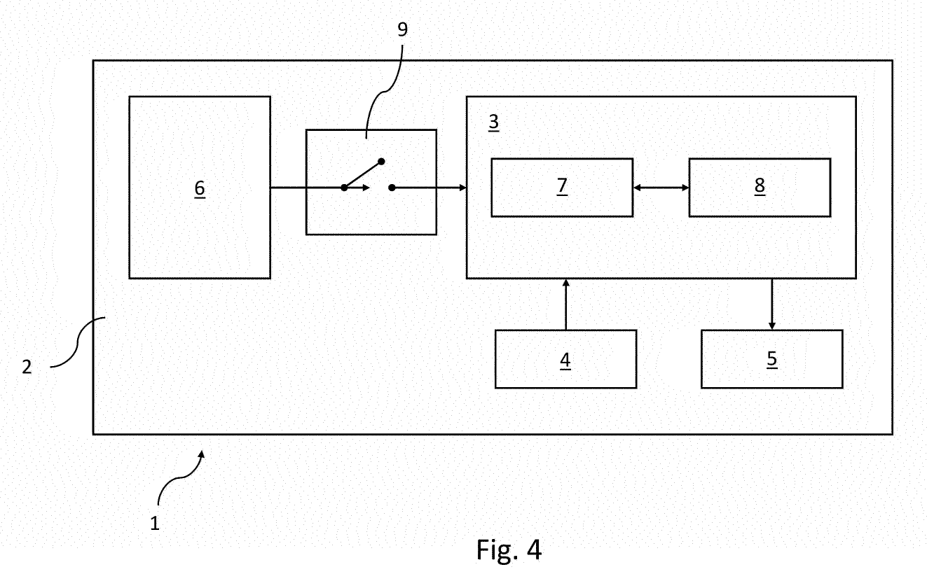

According to an embodiment, in combination with any of those described above, schematically shown in FIGS. 4, 5 and 6, the device 1 can comprise an activation command 9 of the device 1 from an electrical point of view whose actuation, by the user, allows to electrically connect the electronic control unit 3 to the power supply unit 6.

Such activation command 9 can be, for example, a pressure switch, an antifuse or a conductive line which, once interrupted, establishes the electrical connection between the power supply unit 6 and the other components of the device 1, or a removable strip which, once removed, establishes the electrical contact between the power supply unit 6 and the further components of the device 1.

It should be noted that the activation command 9 allows the device 1 to be activated when the device 1 is used for the first time.

In one embodiment, the activation control 9 is flexible, i.e. it has characteristics of thickness (for example up to a lower limit of 20 nanometers) and of manufacturing material which guarantee excellent mechanical flexibility properties.

The activation command 9 can be achieved through the use of printing techniques comprising, but not limited to, printing techniques in printed electronics technology, for example, inkjet printing, flexographic printing, screen printing, etching printing (gravure).

In one embodiment, in combination with the previous one, the electronic control unit 3 is configured to store the value of the count of dispensed doses in the memory module 8 (or in other memory modules with which the electronic control unit 3 can be equipped) performed starting from the activation of the device 1.

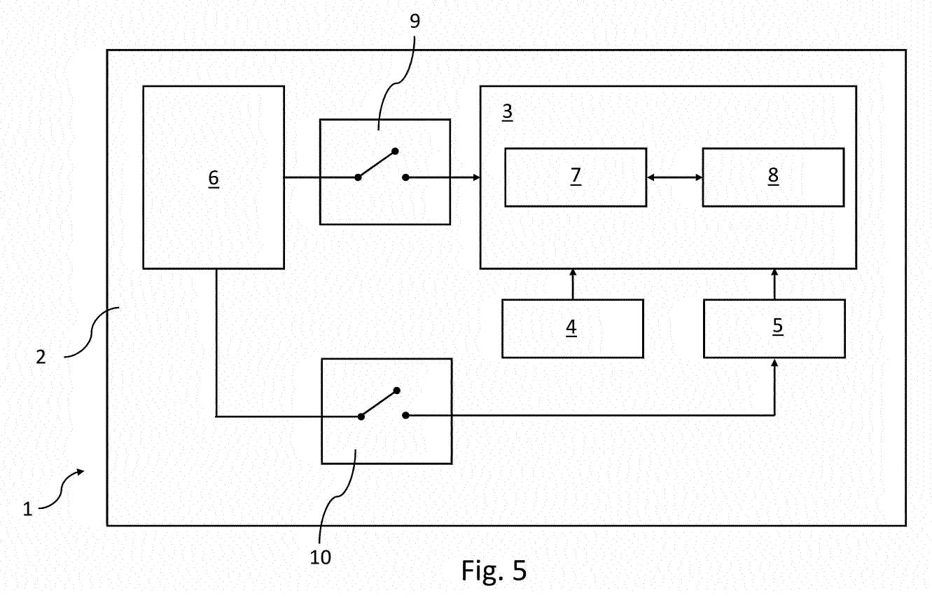

In accordance with an embodiment, in combination with any of those described above and shown in FIG. 5, the device 1 further comprises a command 10 for enabling the information signaling unit 5, the actuation of which, by the user, allows the information signaling unit 5 to be electrically connected to the power supply unit 6.

This enabling command 10 can be, for example, a push button or switch which, once actuated, establishes the electrical connection between the power supply unit 6 and the information signaling unit 5.

It should be noted that the enabling command 10 allows the enabling of the information signaling unit 5, by the user, at any time, upon request.

In one embodiment, the enabling command 10 is flexible, that is, it has characteristics of thickness (for example up to a lower limit of 20 nanometers) and of manufacturing material which guarantee excellent mechanical flexibility properties.

The enabling command 10 can be achieved through the use of printing techniques comprising, but not limited to, printing techniques in printed electronics technology, for example, inkjet printing, flexographic printing, screen printing, etching printing (gravure).

Returning in general to the device 1 (smart label) according to the present invention, it is pointed out that it, or part of it, in one embodiment, in combination with any of those described above, can be combined with a layer overlaid and covered with specific additional graphics, in order to clarify the meaning of the information reported by the information signaling unit.

Such coating layer covered with specific additional graphics can be produced directly on the device 1 or produced separately and then applied with a suitable method.

Furthermore, according to a further embodiment, in combination with any of those described above, the device 1 (smart label) as a whole, or part of it, can be completed with an encapsulating layer capable of limiting or preventing the permeation of specific gases or vapors, for example water vapor and oxygen.

This encapsulating layer can be deposited through coating or printing techniques, or alternatively made separately and laminated on the final device 1.

Such encapsulating layer can be composed, for example, of polymeric materials with a low permeation coefficient for substances of interest, or of particles or “platelets” or “nanorods” of metal oxides, or of metal layers, or of layers of metal oxides, or from particles or layers of silicates or from a combination of one or more of the aforesaid materials.

A part of the device 1, in particular the rear surface of the flexible support substrate 2, as a whole or in part, depending on the specific application, is coated with an adhesive layer to allow adhesion with the target surface(s) of the device for dispensing doses.

This adhesive layer can be achieved not only through the use of a glue or adhesive material, but also, for example, through the following principles or combinations of them: electrostatic adhesion, chemical adhesion, adhesion with “polymer nano brushes”.

In the case of an MDI inhaler, the device 1 (smart label) can be applied, for example, to the container (canister) of the medicinal product or to the actuator dedicated to dispensing doses of the medicinal product.

The device 1 (smart label) object of the present invention, having the function of sequential count of doses dispensed by a device for dispensing doses, can be made with electronic and mechanical technology different from the conventional one, for example belonging to the field of printed electronics and/or organic.

The electronic control unit 3, the sensor unit 4 and the information signaling unit 5 are obtained, for example with techniques in printed electronics technology, of which some examples have been previously supplied, derived from graphic printing technologies and with excellent mechanical flexibility properties (for example plastic organic materials).

These technologies make it possible to realize the functional part of the device 1 in a thin and superficial layer on the chosen flexible support substrate 2, depositing the active materials constituting the components through printing methods (for example, ink jet) in the form of liquid inks, which are subsequently dried by removing the liquid part needed only for the processing.

The electronic components are made with the above procedures through the subsequent deposition, in vertical and/or horizontal geometric structures, of materials of the following type (but not exclusively): conductive polymers (e.g. PEDOT: PSS (poly (3,4-ethylenedioxythiophene) polystyrene sulfonate)), conductive metal inks (e.g. inks containing Ag or Cu nanoparticles, metal-organic complexes, metal “nanowires” or “nanorods”), semiconductors and/or conductors based on carbon derivatives (e.g. carbon nanotubes, graphene), semiconductor metal oxides, conductors or insulators (e.g. Indium-Gallium-Zinc-Oxide, alumina, yttria), semiconductor polymers (e.g. P3HT), small organic molecules (e.g. PCBM ([6,6]-phenyl-C₆₁-butyric methyl ester), Pentacene, F4-TCNQ (2,3,5,6-Tetrafluorine-7,7,8,8-tetracyanoquinodimethane)), dielectric polymers (e.g. Polymethylmethacrylate (PMMA), Polystyrene).

These structures form electronic components suitable for carrying out the functions specified for a smart label such as device 1, including, but not limited to, transistors, diodes, resistors, capacitors, sensors, OLEDs, displays; together with the electrical interconnections necessary for the interconnection of the aforementioned components

With reference now to FIGS. 3, 8 and 9, an example of operation of an electronic device 1 for counting the doses dispensable from a device for dispensing doses of a medicinal, cosmetic or other product is now described, according to an embodiment of the invention.

The device 1 is applied to a container 101 of a Metered Dose Inhaler MDI, as illustrated in FIG. 9.

The sensor unit 4 of the device 1 comprises a pressure sensor (possibly with a threshold) positioned on the upper base of the container (canister) 101 inside which the medicinal product is inserted (FIG. 9).

In order to deliver a dose of medicinal product, the user (patient) exerts on the upper base of the container 101, in correspondence with the pressure sensor 4, a pressure necessary to activate the dispensing valve of the device for dispensing doses in measure equal to or greater than the activation threshold of the sensor (preset and calibrated according to the specific model of MDI inhaler).

The activation of the pressure sensor 4, corresponding to the exceeding of the pressure threshold necessary for dispensing a dose, is detected by the electronic control unit 3 of the device 1 which interprets such activation as dispensing a dose, consequently updating the sequential count of dispensed doses.

The electronic control unit 3 of the device 1, knowing the maximum value of doses that can be dispensed by the device for dispensing doses to which the device 1 is applied, on the basis of the updated value of the sequential count, determines the number of remaining doses to be dispensed and provides this information to the information signaling unit 5 which will provide this information to the user.

As can be seen, the object of the present invention is fully achieved.

Since the components are present only in a thin layer (in some embodiments, less than a micrometer thick) and made of intrinsically flexible materials (for example plastics), the resulting device 1, as already mentioned above, is a smart label having excellent mechanical properties of flexibility that allow, for example, the application on curved surfaces without structural damage.

In addition, manufacturing and assembly techniques can be based on rotary processes, facilitating large-volume production.

Furthermore, the integration techniques of the label are quite similar to the processes already used for the application of graphic labels on existing medical devices, simplifying the introduction of the device 1 as a smart label in existing production lines.

This combined set of advantages not only facilitates the technical integration of the device 1 into current medical devices with existing processes, but simplifies the series of economic and market considerations relating to the costs of introducing such labels in current products, as it enables a low-cost, high-volume type of production process.

In addition, unlike electronic devices based on conventional technology (for example on silicon) which constitute special waste that requires specific and expensive treatment for disposal, the type of technology proposed for device 1 as a smart label, thanks also to the category of functional materials used, can enable strong simplifications in the management of the end-of-life cycle of the final product.

In fact, a large set of organic materials (for example various families of plastics with functional characteristics of the electronic type, some already widely used for applications requiring particular approvals) do not constitute special waste.

In addition, the amount of materials required for implementation is minimal, sometimes less than 1% of the total volume of the label including the flexible backing substrate.

In an embodiment for disposable devices for dispensing doses, the functional part of the device 1 (smart label), composed of electronic circuitry, sensors, display, can be made in a thin and superficial layer which constitutes a proportion less than 5% of the total amount of material of the device 1.

In this example, 95% of the constituent material of the device 1 could be composed of a substrate in polymeric material that can be inserted in common recycling cycles such as, but not limited to, polycarbonate, polyethylene, polyethylene terephthalate, polyethylene naphthalate, polystyrene, paper, or combinations of them.

This feature allows the label to be disposed of in conventional waste management processes, without requiring expensive disassembly and/or special treatment processes.

In other examples, the smart label could be realized with the exclusive use of materials based on carbon or organic polymers, potentially insertable in less complex disposal cycles than the materials commonly used in conventional electronics.

Further embodiments may consist of smart labels in which specific functional parts are detachable from the label, so as to facilitate its disposal and/or reduce the amount of material that requires specific treatment.

Removable components from the smart label can be, for example, the power supply unit 6 (e.g. the battery, the energy harvester), the information signaling unit 5 (e.g. display), the electronic control unit 3 or the functional part as a whole.

In some of its implementations in the case of medical devices, the device 1 performs its functions without coming into contact with the dispensed medicinal product.

This feature is important in the flow of certification and approval procedures for products in this field, as it reduces the need for control, verification and certification activities of such a product.

Furthermore, it should be noted that for the market of devices for dispensing doses, there may exist needs and limitations, even stringent ones, of an economic nature relating to the costs for the addition of the dose counting functions described above with mechanical methods or with the addition of electronics based, for example, on conventional silicon microchips.

For example, in some cases, the cost of adding such functionality may make up the majority of the manufacturing cost of the final device for dispensing doses.

In other cases, the redesign and/or restructuring of the production and supply chain associated with the addition of these functionalities can make an operation suitable for their implementation economically unjustified.

The device 1 (smart label) according to the present invention is designed and implemented in such a way as to overcome these limitations, and can be applied to existing designs without requiring changes to the production process and/or redesign of the product.

In one embodiment, this smart label can be added to existing products in the form of an “add-on” or “retrofit”, so as to add the above functions to the product without any further modification to the label application operation.

In a further embodiment, this smart label can be reusable and/or detachable from one product and reapplicable on a second product, promoting waste reduction and optimizing resource use.

To the embodiments of the device described above, a person skilled in the art, in order to meet contingent needs, can make changes, adaptations and replacements of elements with other functionally equivalent ones, without departing from the scope of the following claims. Each of the features described as belonging to a possible embodiment can be realized independently of the other described embodiments.

1. An electronic device (1) for counting dispensable doses from a device (100) for dispensing doses of a medicinal, cosmetic, or other product, comprising:
a flexible support substrate (2);
an electronic control unit (3) associated with the flexible support substrate (2);
a sensor unit (4) associated with the flexible support substrate (2), the sensor unit (4) being operatively connected to the electronic control unit (3), the sensor unit (4) being configured to detect data representative of an interaction by a user with the device (100) for dispensing doses with which the device (1) is associated, connected to the dispensing of a dose;
an information signaling unit (5) associated with the flexible support substrate (2), the information signaling unit (5) being operatively connected to the electronic control unit (3),
a power supply unit (6) associated with the flexible support substrate (2), the power supply unit (6) being operatively connected to the electronic control unit (3),
the electronic control unit (3) being configured to:
detect an event connected to the dispensing of a dose, by the device (100) for dispensing doses with which the device (1) is associated, based on the data provided by the sensor unit (4);
perform a sequential count of the doses dispensed by the device (100) for dispensing doses with which the device (1) is associated based on data representative of a user's interaction with the device (100) for dispensing doses received from the sensor unit (4);
record the sequential count performed;
determine an information representative of the use of the device (100) for dispensing doses based on the value achieved with the sequential count performed;
provide the information signaling unit (5) with the determined information representative of the use of the device (100) for dispensing doses.

2. A device (1) according to claim 1, wherein the information representative of the use of the device (100) for dispensing doses is an information representative of a number of doses dispensed by the device (100) for dispensing dose, the electronic control unit (3) being configured to assign, to such information, the value achieved by the sequential count performed of the doses dispensed by the device for dispensing doses.

3. A device (1) according to claim 1, wherein the information representative of the use of the device (100) for dispensing doses is an information representative of a number of remaining doses to be dispensed by the device (100) for dispensing doses, the electronic control unit (3) being configured to determine such information as a difference between a maximum value of doses available inside the device (100) for dispensing doses and the value achieved by the sequential count performed of the doses dispensed by the device (100) for dispensing doses.

4. A device (1) according to claim 1, wherein the information representative of the use of the device (100) for dispensing doses is a notification adapted to notify that the doses which can be dispensed by the device (100) for dispensing doses are finishing or are finished, the electronic control unit (3) being configured to determine such information by comparing a difference between a maximum value of doses available inside the dose dispensing device (1) and the value achieved by the sequential count of the doses dispensed by the device (100) for dispensing doses with a minimum alarm threshold value of remaining doses.

5. A device (1) according to any one of the preceding claims, wherein the information signaling unit (5) is configured to provide the user with the information representative of the use of the device (100) for dispensing doses. A device (1) according to any one of the preceding claims, wherein 6. the information signaling unit (5) comprises a display unit.

7. A device (1) according to any one of the preceding claims, wherein the information signaling unit (5) comprises at least one device for triggering a mechanical movement or at least one sound emitter device.

8. A device (1) according to any one of claims 5 to 7, wherein the supply of the information representative of the use of the device (100) for dispensing doses is performed, by the information signaling unit (5), continuously or repeatedly at constant intervals or only upon the dispensing of the dose or at the user's request.

9. A device (1) according to any one of the preceding claims, comprising a command (9) for electrically activating the device (1), the actuation of which, by the user, allows the electronic control unit (3) to be electrically connected to the power supply unit (6).

10. A device (1) according to any one of the preceding claims, further comprising a command (10) for enabling the information signaling unit (5), the actuation of which, by the user, allows the information signaling unit (5) to be electrically connected to the power supply unit (6).

11. A device (1) according to any one of the preceding claims, wherein the electronic control unit (3), the sensor unit (4), the information signaling unit (5), the power supply unit (6), and electric interconnections thereof are as flexible as the flexible support substrate (2).

12. A device (1) according to any one of the preceding claims 9 to 11, wherein the activation command (9) and the enabling command (10) are flexible.

13. A device (1) according to any one of the preceding claims, wherein such a device (1) is completed with an encapsulating layer adapted to limit or prevent the permeation of specific vapors or gases.

14. A device (1) according to any one of the dependent claims, wherein a rear surface of the flexible support substrate (2), as a whole or in part, is coated with an adhesive layer to allow the adhesion to the intended surface(s) of the device (100) dispensing doses.

15. A device (1) according to any one of the preceding claims, wherein the device (1) is a smart label of the stand-alone type.

16. A device (1) according to any of the preceding claims, wherein the electronic control unit (3) is flexible, the electronic control unit (3) being associated with the flexible support substrate (2) by assembling said electronic control unit (3) to the flexible support substrate (2).
